(19) United States
(12) Patent Application Publication
(10) Pub. No.: US 2004/0059199 A1
(43) Pub. Date: Mar. 25, 2004

(54) Wound assessment and monitoring apparatus and method

(76) Inventors: Pamela Sue Thomas, Balesville, IN (US); John M. Savage, Cincinnati, OH (US); Nitin Pant, San Diego, CA (US); Peush Upadhya, West Chester, PA (US); Miles D. Rincker, Indianapolis, IN (US); Scott R. Powell, Fishers, IN (US)

Correspondence Address: Intellectual Property Group, Bose McKinney & Evans LLP, 2700 First Indiana Plaza, 135 North Pennsylvania Street, Indianapolis, IN, 46204, US

(21) Appl. No.: 10/655,412

(22) Filed: Sep. 4, 2003

Related U.S. Application Data
(60) Provisional application No. 60/408,061, filed on Sep. 4, 2002.

(51) Int. Cl. A61B 5/00

(52) U.S. Cl. 600/300

(57) ABSTRACT

A wound assessment and monitoring apparatus and method is provided for monitoring the development of wounds by patients of a health care facility. Surveys are conducted on patients of the health care facility to collect wound information, and wound assessments are performed.

### RELATED APPLICATION

This application claims the benefit of U.S. Provisional Application No. 60/408,061 filed Sep. 4, 2002 and incorporated herein by this reference.

### BACKGROUND AND SUMMARY OF THE INVENTION

The present invention relates to an apparatus and method for assessing the risk of wound development by patients or residents of health care facilities, and monitoring wounds as they are developed by those patients and residents. In particular, the present invention relates to an apparatus and method for efficiently yet non-intrusively collecting and studying wound prevalence, incidence, and occurrence information, and for comparing wound information collected from multiple facilities. The term “facilities” as used herein includes, but is not limited to, hospitals, nursing homes, assisted living facilities, and other institutional facilities, as well as private homes, and other private or semi-private residences, such as retirement homes. The term “patient” as used herein includes, but is not limited to, patients and residents of any such facility.

Current U.S. regulations require certain health care facilities to collect wound and risk assessment data. Presently, many health care facilities collect wound data using printed forms, which are completed manually by the health care worker who is responsible for the patient or resident. Once collected, the wound information may or may not be converted to electronic form and stored.

To help facilities proactively address internal protocols, externally promulgated requirements and regulations (such as JCAHO requirements), and improve the healthcare treatment of patients, there is yet a need for an apparatus and method that permits health care facilities to choose one or more techniques for assessing a patient's risk of developing wounds and allows health care facilities to monitor the prevalence, incidence, and occurrence of wounds in patients at the facilities. Also, it would be desirable to be able to tailor the wound assessment and monitoring procedures to the specific needs or practices of a facility and its patients. Further, it would be preferable for a caregiver to be able to collect the wound information efficiently and reliably, but in a nonintrusive way so as not to cause unnecessary stress to patients. Additionally, it would be advantageous to be able to compare the wound assessment and monitoring results across multiple departments within a health care facility or across multiple health care facilities.

The present invention provides an apparatus for assessing risk and monitoring wounds in patients of a facility. In one embodiment of the present invention a computer program product embodied in a computer readable medium is provided. The computer program product includes instructions that when executed cause a computing device to be operable as a device for monitoring the development of wounds by patients of a health care facility. The computing device operates to display a survey to be conducted on patients of the health care facility. The survey includes at least one wound risk assessment selected from a plurality of possible wound risk assessments. The computing device also displays information about a patient for whom the survey is to be conducted, receives survey data for the patient periodically during the patient's stay at the facility, and displays a risk score for each wound risk assessment in the survey based on the survey data for the patient.

In another embodiment of the present invention, the plurality of possible wound risk assessments includes Braden, Norton, Waterlow, and Hill-Rom risk assessments. The plurality of possible wound risk assessments may include a custom risk assessment for the facility.

In an additional embodiment of the present invention, the computing device is a handheld device. In yet still another embodiment of the present invention, the computing device further operates to display a plurality of available surveys from which a desired survey may be selected. In another embodiment of the present invention, the computing device further operates to store the survey data in a memory.

In a further embodiment of the present invention, the survey is user-definable, or pre-defined by the facility in accordance with the facility's own needs, protocols, and patients. In another embodiment, the survey is configured to be conducted on patients that have a wound (including patients that acquired wounds at the facility and patients who acquired wounds prior to being admitted to the facility), and/or on patients that do not have a wound. In an additional embodiment, the survey tracks the development of wounds by patients who do not have wounds at the time they are admitted to the facility. In yet another embodiment, the support surface being used by the patients (whether or not they have wounds) is monitored.

In accordance with another embodiment of the present invention, a method implemented on a computing device is provided, for studying the development of wounds by patients of a facility. The method includes the step of receiving a plurality of first survey data for a first facility over a network. The first survey data includes wound information and support surface information for patients of the first facility. The method also includes the steps of storing the first survey data in a memory, and analyzing the first survey data to determine whether there is a relationship between the wound information and the support surface information.

In an alternative embodiment of the present invention, the wound information includes an indication of whether patients have wounds. In yet another embodiment of the present invention, the support surface information includes the name of a support surface.

In an additional embodiment of the present invention, the method includes the step of communicating the results of the analyzing step to a person having a relationship with the facility. In yet another embodiment, the entity is a person affiliated with a support surface manufacturer.

In still another embodiment of the present invention, the method further includes the step of receiving a plurality of second survey data for a second facility over a network. The second survey data includes wound information and support surface information for patients of the second facility. The method also includes the steps of storing the second survey data in a memory, and comparing the second survey data to the first survey data.

In an additional embodiment of the present invention, the first and second survey data include assessment values, and the method includes the step of averaging the assessment values of the first and second survey data.

In a further embodiment, the first survey data includes support surface information for patients that do not have a wound. In another embodiment, a wound incidence percentage is determined for each support surface in use by patients of the facility. In yet another embodiment, the number of wounds is determined by wound stage and by support surface.

In accordance with another embodiment of the present invention, a computer program product embodied in an electronic storage medium is provided. The computer program product includes instructions that when executed cause a computing device to be operable as a device for monitoring the incidence of wounds in patients of a health care facility. The device operates to receive a plurality of wound information for a grouping of patients of the facility. The wound information includes an indication of whether a patient has a wound. The device computes a wound incidence percentage for the grouping of patients, and communicates the wound incidence percentage to a person associated with the facility.

In another embodiment of the present invention, the wound incidence percentage is computed over a time period equal to the average patient stay. In an additional embodiment of the present invention, the wound incidence percentage is computed for each patient for a time period beginning on an admission date and ending on a discharge date. In yet another embodiment of the present invention, the computing device operates to receive information about an activity level of the facility. In a further embodiment of the present invention, the computing device operates to analyze the activity level information and the wound incidence percentage. In another embodiment of the present invention, the computing device operates to conduct an occurrence study.

The apparatus of the present invention permits the development of wounds by patients to be monitored. Whether the wound was present at the time of admittance to the healthcare facility or was acquired during his or her stay at the facility can be tracked. Further, the responsiveness of a patient or group of patients to particular forms of treatment can be tracked through incidence studies. In addition, the quality of care being provided by health care workers in a facility or a department or division of a facility can be assessed through occurrence studies.

In an illustrated embodiment of the present invention, a bed, therapy surface, or other support surface being used by a patient is monitored and compared with support surfaces used by other patients in the same department, with patients in other departments in the same health care facility, or with patients in other facilities. This information may be used, for example, to determine whether a change to the support surface being used by the patient could be beneficial to the patient.

Further, in accordance with another embodiment of the present invention, support surface information is collected independently of or in connection with the wound information. In other words, an evaluation of a patient's support surface is performed regardless of the technique or assessment criteria used to monitor wounds in this embodiment.

As used herein, the term “support surface” includes, but is not limited to, mattresses, pads, coverlets, and portions thereof; sleep surface systems (including a surface or a surface in combination with a frame) such as the Hill-Rom® ZoneAire® brand systems; therapy surfaces (including dynamic, airflow, low airloss, and pressure relief therapy surfaces) such as the Hill-Rom® Acucair® brand surfaces; air fluidized therapy units (including units that provide air fluidized therapy alone or in combination with low airloss therapy) such as the Hill-Rom Clinitron® brand units; mattress replacement systems (including systems that provide dynamic therapy alone or in combination with pressure relief) such as the Hill-Rom® V-Cue® systems; mobile therapy units (including units that provide low airloss and/or other pressure relief therapies) such as the Hill-Rom Flexicair Eclipse® brand units; and adjustable-width mattresses; all of the above whether provided with or without frames, covers, or coverlets. In addition, the term, “support surface” includes, but is not limited to, portions of any of the above surfaces, units and systems, including independent zones (alone or in combination with other zones) such as pressure relief zones provided to accommodate a patient's heels or other areas of the body that may be at risk of developing pressure ulcers; and side bolsters. Further, the term “support surface” includes any surface suitable for supporting persons, including solid and porous surfaces (made of foam or three-dimensional fiber material, for example), surfaces made up of a plurality of layers, and filled surfaces such as inflatable bladders.

In another illustrated embodiment of the present invention, non-healthcare-facility entities are provided access to certain portions of the collected data. Currently, there are laws and regulations that may restrict or prohibit access by certain such entities to personally-identifiable health information. Therefore, in such instances, patient-identifying information is generally removed from the data prior to access by outside entities.

For example, the manufacturer of a bed or other support surface may be given access to the data in order to study and compare survey results for multiple healthcare facilities based on the support surface in use. This analysis may prove valuable to such a manufacturer in that the manufacturer may be able to more quickly and easily identify the need for changes to existing products or to develop new products.

Also, the manufacturer may communicate product recommendations to healthcare facilities or providers based on its analysis of survey results.

### BRIEF DESCRIPTION OF THE DRAWINGS

FIGS. 1a-1f show block diagrams of alternative embodiments of the apparatus of the present invention;

FIG. 2 shows a flow diagram of a process in accordance with the present invention;

FIG. 3 shows a flow diagram of another process in accordance with the present invention;

FIGS. 4a-4c show screen displays of one embodiment of the process of creating a survey in accordance with the present invention;

FIGS. 5a-5b show screen displays of one embodiment of the process of adding patients to a survey in accordance with the present invention;

FIGS. 6a-6k show screen displays of one embodiment of the process of conducting a survey in accordance with the present invention; and

FIG. 7 shows an example of an incidence report in accordance with the present invention.

### DETAILED DESCRIPTION OF THE DRAWINGS

The present invention provides an electronic survey software application for collecting and monitoring wound information for patients in a health care facility. While the drawings show a particular application of the present invention directed to pressure ulcer surveys, it is understood that the present invention can be readily adapted to collect and monitor data for various other types of wounds.

In accordance with one illustrated embodiment of the present invention, a computer program product operates a computing device to permit health care facilities to create customized wound assessment surveys. A facility may create one or more such surveys for different patient populations. In accordance with another embodiment of the present invention, a computer program product operates a computing device to enable caregivers to perform wound surveys during the regular course of a patient's treatment or therapy routine, either manually or by using a suitable computing device, such as a microcomputer, laptop computer, personal digital assistant (PDA), cell phone, or other electronic device with computing capabilities. In the illustrated embodiment, suitable computing devices include personal computers running a Windows® or similar operating system and handheld devices running a Palm™ or similar operating system.

In accordance with yet another embodiment of the present invention, a computer program product operates a computing device to analyze the survey results and/or compare the survey results with other survey results; for example, to conduct prevalence, incidence, or occurrence studies. The results of these studies can be used in a variety of ways by a caregiver, health care facility, or other entities, such as hospital equipment manufacturers, to improve patient care, as further described below.

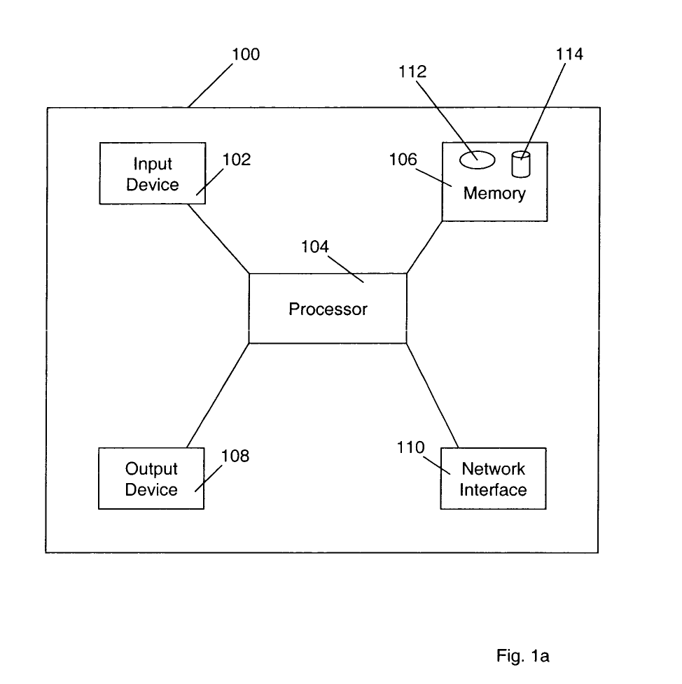

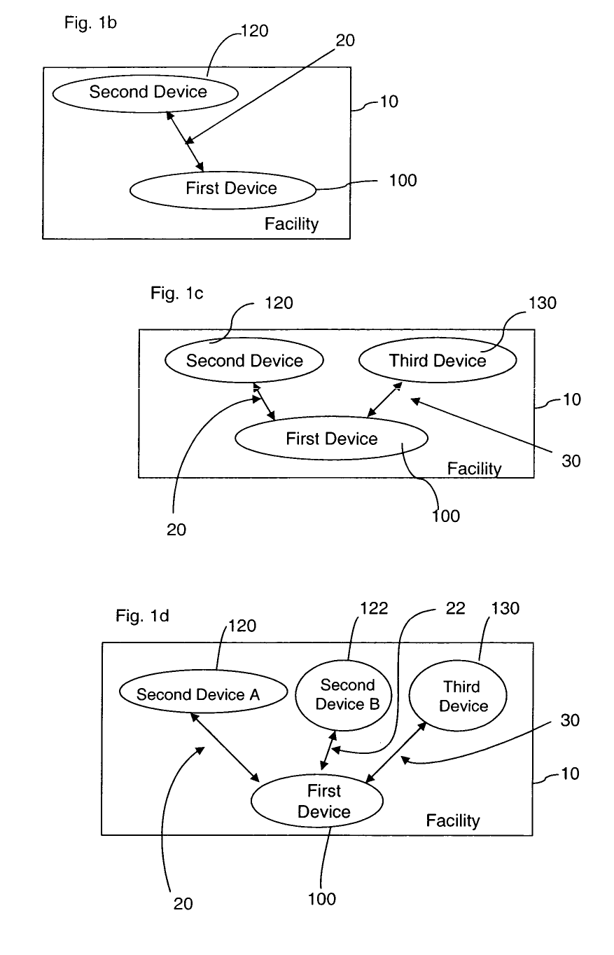

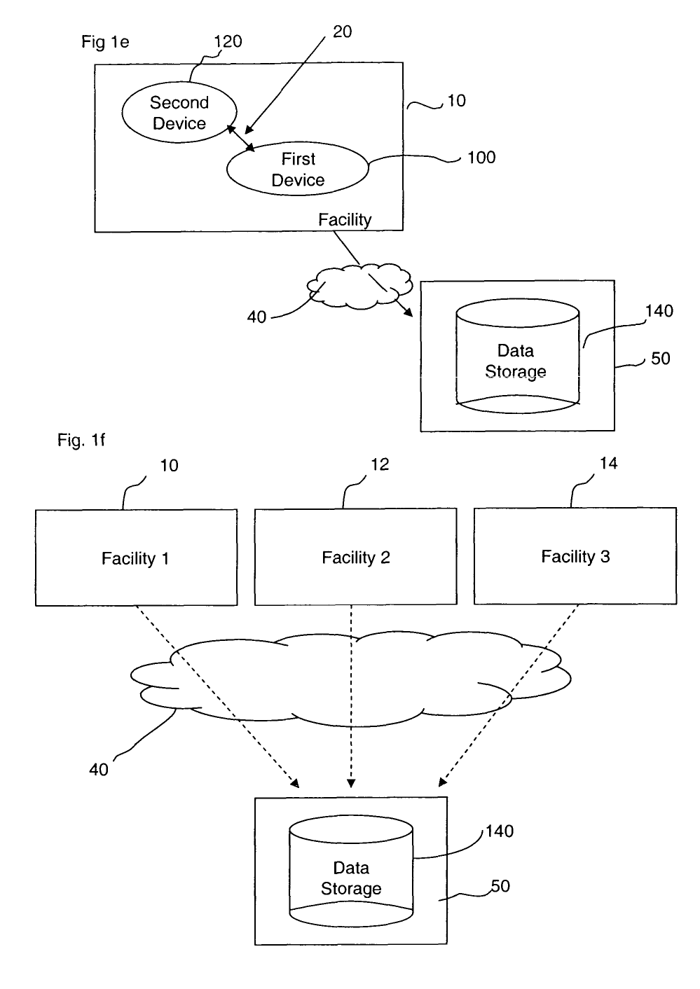

FIGS. 1a-1f show alternative embodiments of an apparatus configured in accordance with the present invention. Those skilled in the art will appreciate from these drawings that still other embodiments not shown will fall within the scope and spirit of the present invention.

FIG. 1a shows a first embodiment of an apparatus configured in accordance with the present invention. The apparatus of FIG. 1 is a first computing device 100. Included in first computing device 100 is an input device 102, a processor 104, at least one memory 106, and at least one output device 108 coupled together via electrical circuitry or other suitable coupling means. Optionally, a network interface 110 is also provided, for connecting first computing device 100 to a communications network.

Input device 102 is, for example, a computer mouse, electronic stylus (or “pen”), touch screen, keyboard, keypad, microphone/voice recognition system, scanner, or other suitable apparatus for entering information in electronic form known in the art.

Processor 104 is one or more suitable microprocessors well known in the art, which processes data 114 and computing instructions 112 received via input device 102 or stored in memory 106. Processor 104 executes computing instructions 112 to perform processes such as those described below in accordance with the present invention.

Memory 106 is a volatile or non-volatile storage media, such as ROM, RAM, flash memory or other media, suitable for storing data 114 and instructions 112. As is well known in the art, memory 106 may include a plurality of distributed memory coupled via electrical circuitry.

Output device 108 is a computer monitor, LCD display, flat-screen display (for example, gas-plasma, active-matrix or ELD display), or similar device (including a display of a personal digital assistant or other handheld or portable device) suitable for display of visual information, and/or a speaker or other suitable device for outputting audio information, as known in the art.

Data 114 may be stored in one or more databases or data warehouses in memory 106. As is well known in the art, a database is an organized body of information, such as a collection of data arranged for search and retrieval, and may include structured tables or records, multiple computer files, or a single file. In the illustrated embodiment, a relational database product such as SQLServer, Oracle, or Access is used. One of ordinary skill in the art would readily understand, however, that other suitable means for storing and retrieving data 114 may be used equally effectively.

In the embodiment of FIG. 1a, wound care surveys are created using first computing device 100. Preferably, the surveys are also conducted using first computing device 100, but it is understood that surveys may be conducted manually, e.g., using paper forms. Wound survey results are analyzed and reviewed using first computing device 100. The processes of creating, and conducting surveys, and analyzing and reviewing survey results, are described more fully below.

FIG. 1b shows a second embodiment of an apparatus configured in accordance with the present invention. The apparatus of FIG. 1b includes a first computing device 100 and a second computing device 120 connected by a network 20. Network 20 is a communications network for communicating information electronically, for example, implemented using a cable, telephone, DSL, wireless, or other suitable medium known in the art.

In the embodiment of FIG. 1b, first computing device 100 is a “server” computer such as a microcomputer or personal computer, or, depending on the volume of surveys to be created and/or survey data to be collected, a computer with greater storage and/or processing capacity, such as a minicomputer or mainframe. One or more first computing devices 100 are used by one or more authorized persons associated with facility 10 to create customized wound assessment surveys for facility 10, as described in greater detail below. Once a survey is created, it is transferred or uploaded to second computing device 120 via network 20.

Second computing device 120 is a computing device, as described above, preferably, a handheld device, such as a PDA. In the embodiment of FIG. 1b, a portion of programming instructions 112 are stored in a memory 106 on second computing device 120 so that wound assessment surveys can be conducted using second computing device 120. In the illustrated embodiment, the ‘C’ and ‘C++’ programming languages are used to code the programming instructions for operating a handheld device in accordance with the present invention and to transfer data to and from the handheld device, but it is understood that any other suitable programming languages may also be used.

An installation computer program is executed to install the wound assessment software program and its associated databases on second computing device 120. If a PDA is used as second computing device 120, the installation program performs the installation using an innovative “single-sync” single step process. In comparison, other known installation programs, for PDA applications in particular, require two steps: one step to install a software application and then a second step to install the associated databases. Of course, one of ordinary skill in the art will appreciate that any suitable installation programs may be used (whether using one or two steps).

The single sync install method “prepackages” the data and software application during installation, as follows. During installation of the application on the handheld device, a variable is set within a computer program structure known as a “conduit” that directs the stream of data that would normally go to a handheld device to a class inside the conduit that writes files locally to the microcomputer from which the installation is being performed. A “behind the scenes” synchronization is then performed. The conduit reads data from a database as it normally would during a synchronization, but the stream of data that would be sent to a connected handheld device is instead written to a binary handheld-device database file (e.g., file type pdb) on the microcomputer. This process is transparent in that the user of the installation program is unaware that it is happening. The pdb files are then copied to an install directory on the microcomputer. The application for the handheld version of the software application is also moved to the install directory. As a result, when the installation program completes, the user is required to synchronize his or her handheld device only one time. During this single synchronization, both the application and the data files are moved from the install directory to the user's handheld device.

Preferably using the single-sync process described above, second computing device 120 receives a pre-defined survey from first computing device 100, over network 20. Of course, if second computing device 120 is a wireless device, at least a portion of network 20 is a wireless network.

In the embodiment of FIG. 1b, the conduit is used to control the exchange of data between first computing device 100 and second computing device 120, particularly if second computing device 120 is a PDA. The conduit is used to synchronize data between a PDA 120 and a first computing device 100. In the illustrated embodiment, the time stamp associated with data on PDA 120 is synchronized with the clock of first computing device 100 to ensure data integrity. The conduit keeps track of the most recent version of data across multiple devices, each of which has its own database. For example, in the case where one health care provider completes a survey for a patient on a computing device 120, and then a second health care provider completes another survey for the same patient on another computing device 122 (see, e.g., FIG. 1c described below), the conduit will determine the order in which the surveys were conducted and store the survey data in the proper order.

Data collected by second computing device 120 is eventually transferred to first computing device 100, where it is stored in memory 106. In the illustrated embodiments, suitable encryption algorithms known in the art are used to protect the confidentiality of personally-identifiable health information, such as first name, last name, and medical record number. It will be understood by those skilled in the art that personally identifiable information, and other sensitive data, may be protected from unauthorized access by encryption or other means known in the art.

As will be appreciated by one of ordinary skill in the art, first computing device 100 and second computing device 120 may be selected in accordance with a particular facility's requirements, including the number of different surveys to be conducted and the number of patients to be surveyed.

FIG. 1c shows a third alternative embodiment including a third computing device 130. In this embodiment, third computing device 130 is a microcomputer, such as a personal computer, which is in data communication with first computing device 100 via communication link 30. However, in other embodiments, third computing device 130 may also be a handheld, PDA, or other suitable computing device. Communication link 30 is an internal or external communications network of facility 10.

In FIG. 1c, third computing device 130 is configured for use by one or more authorized persons, such as a system administrator, to perform administrative activities, such as managing user accounts. In this way, the administrative functions may be conducted from one or more computing devices that are in addition to the computing device that stores the collected survey data. While not specifically shown in FIG. 1c, it is understood that one or more third computing devices 130 may be used, if, for example, more than one person is assigned to perform administrative tasks. Alternatively, surveys may be created and customized, and reports may be generated, using third computing device 130.

A fourth alternative embodiment is shown in FIG. 1d. The system configuration of FIG. 1d is similar to that of FIG. 1c, except that in the embodiment of FIG. 1d, multiple second computing devices 120, 122 may be used to collect survey data. As many second computing devices 120, 122 as are necessary may be used, e.g., one such computing device may be provided for each health care worker who is assigned to treat or care for patients in the facility. Also, as discussed above, second computing devices 120, 122 may be either handheld devices, PDAs, or personal computers, or similar suitable devices known in the art.

A fifth alternative embodiment is shown in FIG. 1e. In the embodiment of FIG. 1e, the wound assessment and monitoring survey system of facility 10 is connected to a central data repository at another location 50, data storage 140, via a secure external network 40. In this embodiment, survey data is collected using first computing device 100 and/or second computing device 120, and transmitted from facility 10 to data storage 140 via secure network 40, where it can be compared against other data for facility 10, or against data from other facilities. In the illustrated embodiment, personally-identifiable data patient is removed prior to the transmission of data to data storage 140, to further protect the confidentiality of such information.

In the embodiment of FIG. 1e, network 40 is a public or private network that connects at least two entities, facility 10 and location 50. For example, network 10 may be a secure portion of the Internet, as is well known in the art. Location 50 is, for example, a computer system at another facility in an affiliated network of healthcare facilities that includes facility 10. Location 50 may also be a computer system of a third-party hosting service. Further, location 50 may be a computer system of a non-healthcare-facility entity, such as a manufacturer or vendor of healthcare products involved in the treatment of wounds, for example, a manufacturer of patient support surfaces.

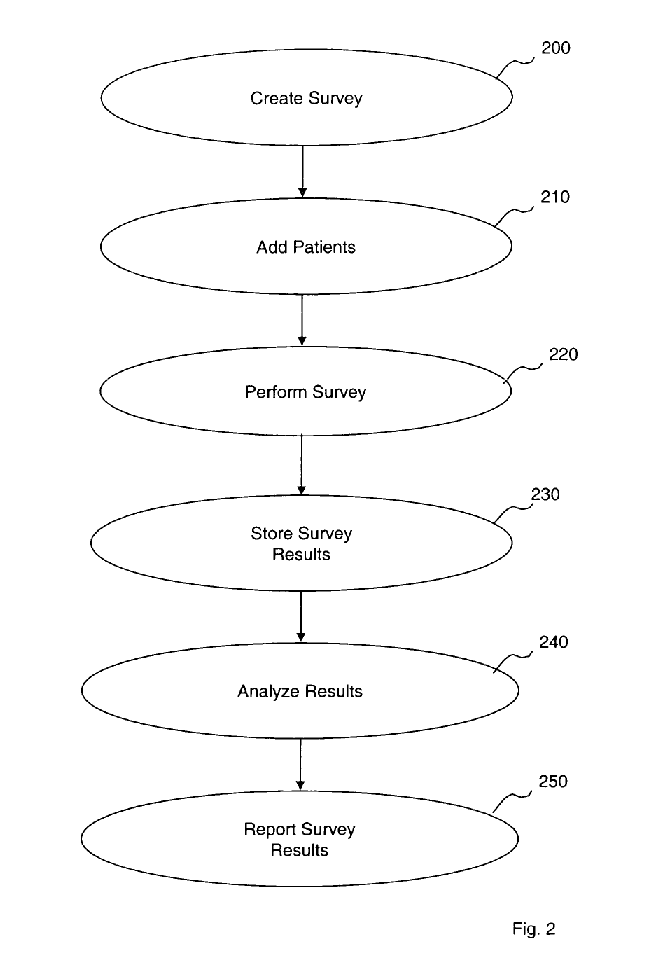

FIG. 1f illustrates a sixth alternative embodiment of the present invention, wherein multiple facilities 10, 12, 14 are connected to a location 50 that includes a data storage 140. Each of facilities 10, 12, 14 transmits collected survey data to location 50 via a secure network 40. As mentioned above, personally-identifiable patient data is generally removed prior to such transmission. Survey data is received from one or more facilities 10, 12, 14 by location 50 and stored in data storage 140. A computer program product installed at location 50 (not shown) or on first computing device 100 or second computing device 120 accesses and analyzes the survey data and generates reports showing the survey results and results of analysis, as discussed further below FIG. 2 shows a flow diagram of a process for assessing and monitoring patient wounds in accordance with the present invention. At step 200, a customized survey for a facility is created. In the illustrated embodiment, a customized survey is defined by a person or software process that is an authorized actor for the facility, e.g., an administrator. The administrator's authority is verified using password or other security routines well known in the art.

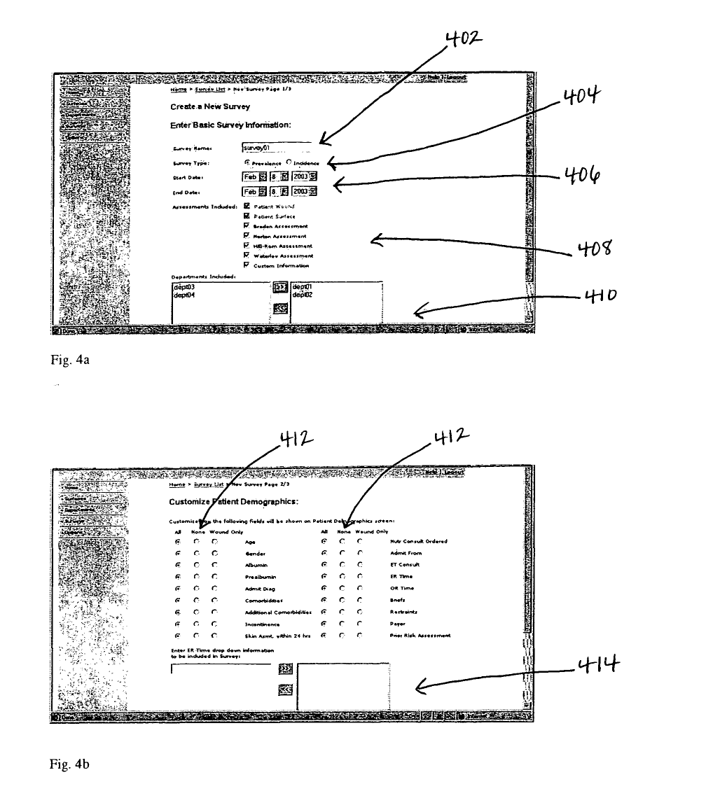

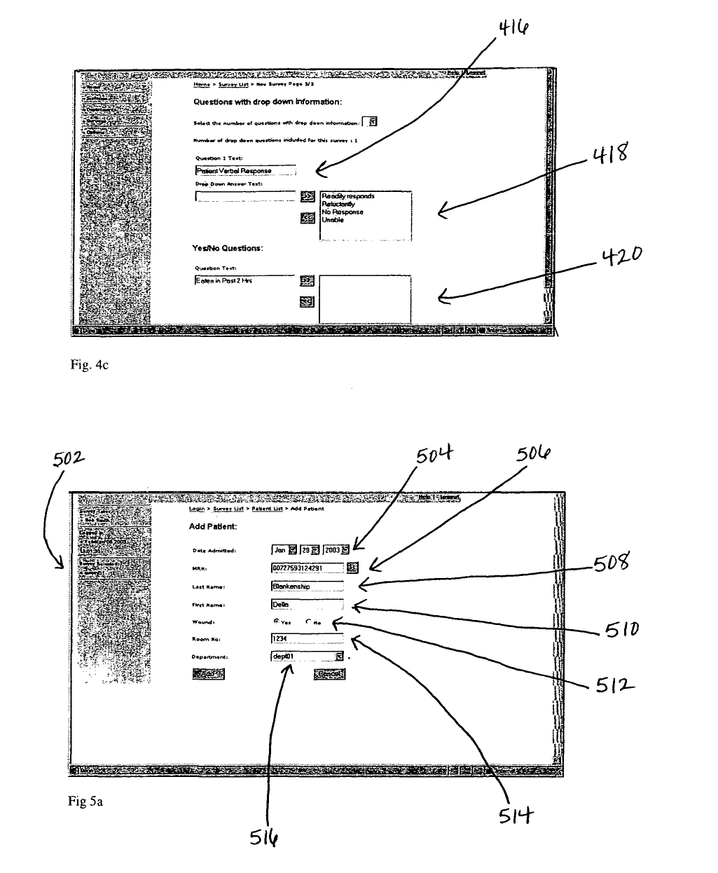

To create a survey, an authorized user, such as an administrator, responds to questions and prompts displayed on a user interface of a computing device 100, 120, 130, described above. FIGS. 4a-4c show example screens for creating a survey. As shown in FIG. 4a, the authorized user inputs a name for the survey at input area 402, survey type (as shown, either “prevalence” or “incidence”) at input area 404, the survey start and end dates at input area 406, and selects the assessment(s) to be included in the survey at input area 408. As shown, the available assessments include “patient wound”, “patient surface”, “Braden Assessment”, “Norton Assessment”, “Hill-Rom Assessment”, “Waterlow Assessment”, and “Custom Information”, however, it is understood that additional or alternative assessment types may also be included. As shown in FIG. 4a, all of the available assessment types are selected. Below the list of assessments is an input area 410, where the user may specify particular groupings of patients to be the subjects of the survey. As shown in FIG. 4a, the available groupings include two hospital departments shown on the left, and two hospital departments have been specified, as indicated on the right. However, it is understood that any other grouping of patients may be specified in alternative embodiments. For example, a grouping of patients could be defined by age or other demographic criteria.

As shown in FIG. 4a, the available survey types 404 are “prevalence,” and “incidence.” Another survey type, “occurrence,” is not specifically shown in FIG. 4a but is also available. It is understood that additional or alternative survey types may be specified as may be desired by a particular facility.

A prevalence survey is used to take a one-time assessment of a wound, for an individual patient, a grouping of patients, or all patients in a facility. In the illustrated embodiment, a prevalence survey is a “snapshot” of wound data. For example, a prevalence survey would collect information that would enable a facility to determine, on a given day, how many or what percentage of patients, have reported having at least one pressure ulcer or other wound. In the illustrated embodiment, prevalence is calculated by dividing the total number of patients with wounds by the total number of patients in the study (with or without wounds). Prevalence is alternatively determined by excluding patients with wounds in certain stages (such as stage I wounds) (discussed below), and by including only those patients who have developed wounds since arriving at the facility.

An incidence survey is an ongoing survey that monitors the condition and status of patients' wounds over time. Incidence surveys are useful, for example, to deter mine if patients without wounds acquire wounds over a period of time, if the patients are improving, if different therapies or treatment surfaces affect the wound condition, or whether wounds are being acquired during the hospital stay. In the case of incidence surveys, assessments are periodically repeated, for example, on a daily basis, for a predetermined period of time. For example, an incidence survey may be conducted for the duration of the average length of patients' stay at a facility, or, for each patient, for the period from admittance to discharge, e.g., on the date of admission and then each day thereafter until discharge.

In the illustrated embodiment, an incidence percentage is calculated by, at each sample time during the duration of the survey (e.g., daily) dividing the total number of patients having pressure ulcers by the total number of patients surveyed at the beginning of the study that did not have pressure ulcers. As with prevalence studies, selected wound stages can be excluded and patients who did not acquire wounds at the facility can also be excluded.

An “occurrence” study monitors, for a grouping of patients, the occurrence of wounds in view of the number of patient days during a time period. Occurrence studies are useful to evaluate the condition of the patients in view of the activity level of the hospital or department being monitored. The activity level is based on the number of patients admitted or treated by the department during the monitoring time period. For example, if the number of pressure ulcers reported increases, but the department activity level also increases, the department staff performance may be considered satisfactory given its increased activity level.

In the illustrated embodiment, occurrence is calculated by dividing the number of new wounds acquired during the study period by the number of patient days in the study period, for example, the number of new pressure ulcers appearing during the study divided by the number of patient days during the study period. The number of patient days differs from the number of days in the study period in that the number of patient days includes, for each patient, only those days on which the patient is under the facility's care or supervision. For instance, if a patient is discharged during the study, for that patient, the number of days from the date of discharge to the end date of the study would not be included in the total number of patient days in the study.

The wound risk assessments 408 selected to be included in a survey are typically analyses used in the industry to help a caregiver or healthcare provider determine whether a particular patient is at risk for developing a wound. In general, the available risk assessments will be familiar to caregivers and health care providers. The Braden risk assessment is well known in the industry and collects wound-related patient data in the following categories: sensory, moisture, activity, mobility, nutrition, and friction. The Norton risk assessment is also well-known and collects wound-related patient information in the following categories: general, mental, activity, mobility and incontinence. The Waterlow risk assessment is a type of assessment used primarily in Great Britain. The Waterlow risk assessment tracks a patient's gender, size (e.g., height/weight/build), continence, skin type, mobility, appetite, whether the patient has had terminal cachexia, cardiac failure or peripheral vascular disease or anemia, whether the patient is a smoker, whether the patient has had surgery below the waist or near the spine, whether the patient has spent an extended period of time in the operating room (e.g., more than 2 hours), whether the patient has been prescribed steroids, anti-inflammatories, or other medications, and whether the patient has any neurological deficit. The Hill-Rom risk assessment collects wound-related patient information in the following categories: continence, mobility, nutrition, contractures, skin integrity, and old/previous pressure ulcer scores.

A given survey may utilize one or more of the available risk assessments. For example, a survey may contain only the Norton risk assessment, or both the Braden and Norton risk assessments. Each of the risk assessment methods calculates a risk score based on the responses to the survey criteria, and gives an assessment of the patient's risk of developing wounds based on the risk score. For example, using the Braden assessment, depending on the responses entered by the survey taker in the above-described categories, the patient is determined to be at no, low, moderate, high, or very high risk of developing wounds.

A surface assessment collects information relating to the support surface used by the patient (e.g., surface name, manufacturer, model, start date on surface, and end date on surface). A wound assessment collects information relating to a particular wound of a patient, such as its stage, type, source (e.g., if acquired while in the facility), location and/or sub-site on the body, documentation and outcome. In the illustrated embodiment, once patient, wound, and surface information is collected, it is immediately available for display on the computing device used to collect the information (i.e., the handheld device or microcomputer).

Referring back to step 200 of FIG. 2, in creating a survey, a facility specifies which patient demographics it desires to monitor. FIG. 4b shows an example screen for selecting patient demographics to be included in a survey. The available patient demographics to be monitored include age, gender, albumin level, prealbumin level, admission diagnosis, comorbidities, incontinence, skin assessment information, nutrition consultation information, information about where the patient is admitted from, information about an ET consultation, date/time admitted to emergency room, date/time admitted to operating room, information about briefs (diapers) or restraints in use, information about the patient's primary payor, and information about any previous risk assessments. As shown in FIG. 4b, radio buttons 412 are used to select whether to track each demographic for all patients, for no patients, or only for patients that have a wound. At the bottom of the screen shown in FIG. 4b, an input area 414 is provided to permit additional information relating to emergency room or operating room times to be recorded. It is understood that when a survey is conducted, the survey information may be requested and input in any suitable form, including audio as well as visual form.

In creating a survey, a caregiver or facility may also specify additional “custom” questions to be included in survey. FIG. 4c shows an example of a screen that may be used to define additional survey questions. The name of the new survey question is input at input area 416. The available choices for responding to the survey question are specified in input area 418. In this example, the choices for responding to the new survey question are limited to the answers listed in input area 418. Input area 420 is used to define new survey questions for which the answer is either “yes” or “no.” As will be understood by one of ordinary skill in the art, other types of survey questions may also be created, although not specifically shown in the drawings.

Once a new survey is created, one or more patients are selected to be assessed during the survey at step 210 of FIG. 2. In the illustrated embodiment, patients are selected to participate in a survey before the survey is conducted. Alternatively, patients are selected at the time the survey is conducted. FIG. 5a shows an example screen for adding a patient to a survey. The selected survey, to which one or more patients will be added, is indicated in display area 502. The current survey taker and date/time connected are also shown in display area 502. Basic information about the patient is entered, including date of admittance, medical record number (MRN) or other non-personal unique identifier, last name, first name, whether the patient currently has a wound, the patient's room number in the facility, and the department to which the patient is assigned within the facility, at input areas 504, 506, 508, 510, 512, 514, and 516, respectively. A unique identifier can be automatically generated by selecting the “R” button. Alternatively, it is manually input by typing an identifier in the space to the left of the “R” button. Selecting the “add” button 518 adds the patient to the survey, while selecting the “cancel” button completes the “add patient” function without adding the patient to the survey.

When at least one patient has been added to a survey, a screen such as is shown in FIG. 5b displays summary information about the patient or patients added to the survey. As shown in FIG. 5b, a search capability 522 is provided to allow a user to search for information about a particular patient, for example, by the patient's last name, room, department, or other criteria. At display area 524, summary patient information is shown for all patients in the selected survey (indicated in display area 502), including, the patient's name, date admitted to the facility, whether the patient has been discharged, the patient's unique identifier or MRN, department, room, whether the patient has a wound, and the status of the patient's survey (i.e., whether the survey has been completed for the patient). As shown, a series of buttons are provided to enable a user to take various actions with respect to the patients in the survey. The “done” button 526 is selected to indicate to the system that the user is finished adding patients. The “add” button 528 is selected to add additional patients to the survey. The “edit” button 530 is selected to modify information about an existing patient in the survey. The “delete” button 532 is selected to delete a patient from the survey. The “discharge” button 534 is used to indicate that a patient has been discharged from the facility and thus any currently incomplete surveys should not be completed. The “assessments” button 536 is selected to view the status of assessments for a patient, or to complete additional assessments in the survey for a patient.

Once a new survey is defined, it may be conducted by a caregiver or health care provider, either manually or using a first or second computing device 100, 120, 122, as discussed above.

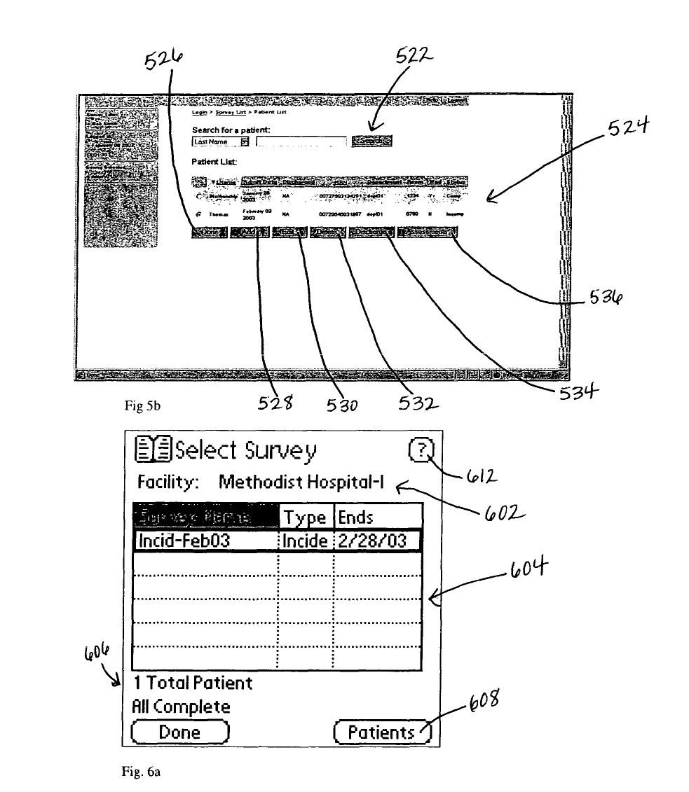

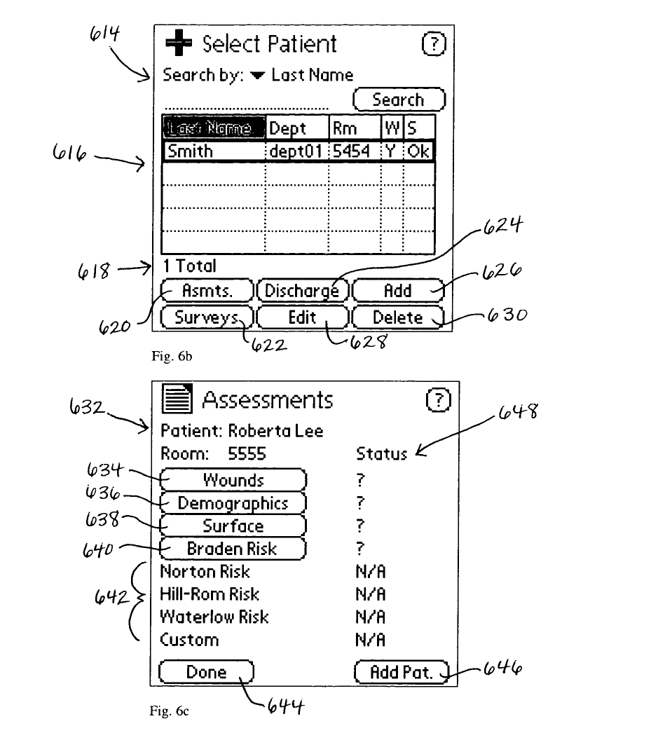

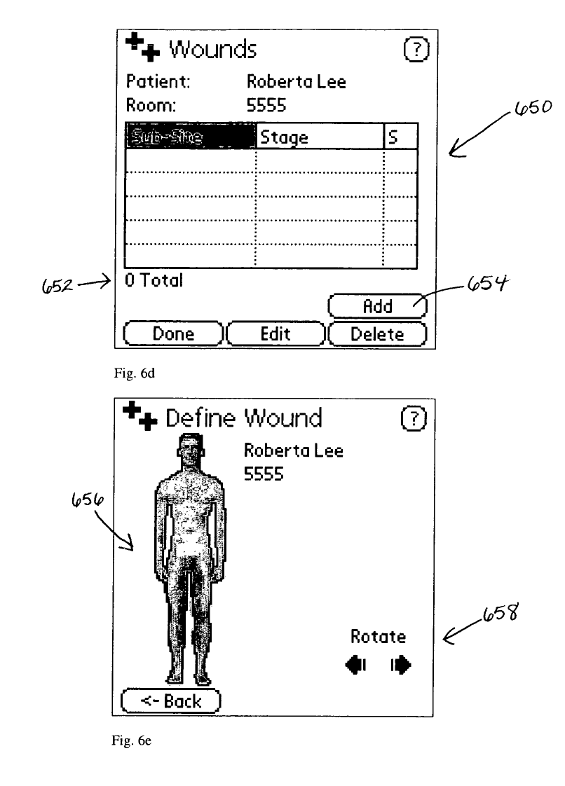

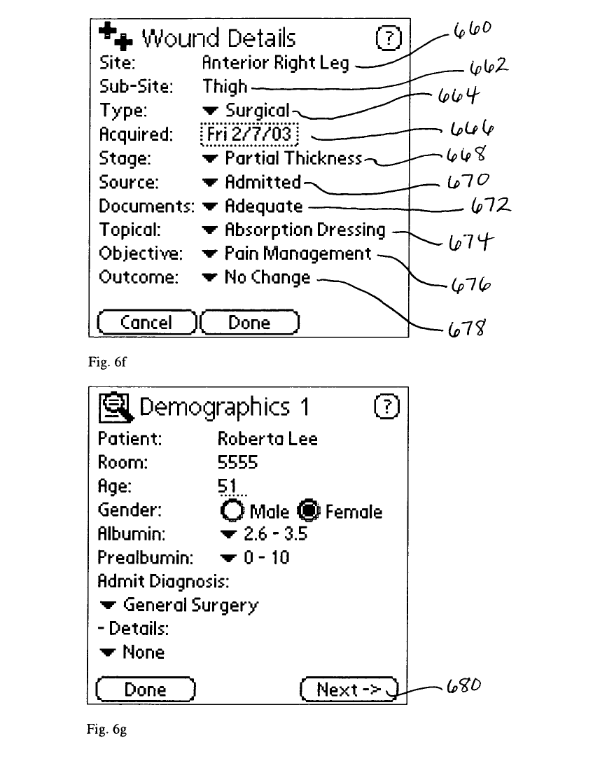

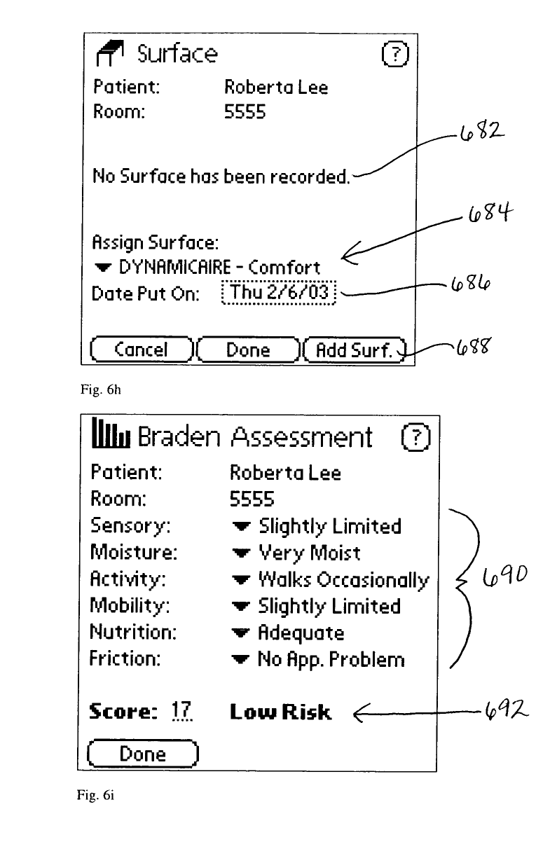

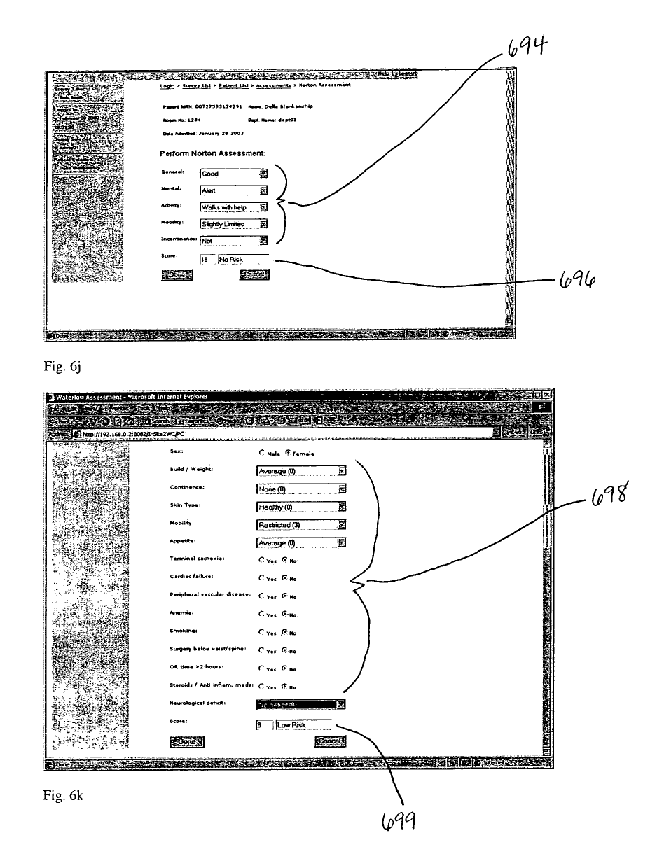

At step 220 of FIG. 2, a wound survey that has been created in step 200 is performed on patients added to the survey in step 210. When a caregiver or health care provider (“survey taker”) successfully connects to the electronic survey application, i.e., by entering a user name and password, a list of available surveys for facility and associated with the survey taker are displayed. As illustrated in FIG. 6a, the facility name is displayed at block 602. The survey name, survey type, and date information for each available survey is displayed in table 604. Since in the illustrated example, the survey type is “incidence”, an end date is specified. If the survey type were “prevalence”, a date on which the survey is scheduled to be performed would be shown instead of an end date.

A list of patients associated with the survey user is also displayed, or the survey user may search for a specific patient. The survey taker selects the appropriate survey and patient. The text at block 606 indicates the number of patients involved in the survey and the number of patients for whom the survey has been completed. The “patients” button 608 is selected to view and/or edit information about patients in the survey. The “done” button 610 is selected when the survey taker does not wish to conduct any more of the available surveys. The “help” button 612 provides online help instructions to the survey taker, to explain, for example, how to conduct a survey using the online system.

In the illustrated embodiment, a screen such as is shown in FIG. 6b is displayed in response to a survey taker's request to view or edit patient survey information (e.g., by selecting the patients button 608 of FIG. 6a). As shown in FIG. 6b, a list of all of the patients assigned to the selected survey is provided in table 616. A search utility is provided at block 614, so that a survey taker can locate a particular patient within table 616. The assessments button 620 is selected to perform an assessment on a patient selected from table 616. The discharge button 624 is selected to discharge a patient as discussed above. The add button 626 is selected to add a new patient to the survey. The surveys button 622 is selected to return the survey taker to the list of available surveys, e.g., FIG. 6a. The edit button 628 and delete button 630 when activated allow the survey taker to edit information for a selected patient or delete a selected patient, respectively.

If a survey taker desires to conduct a survey for a specific patient, the patient's name is selected from table 616 and the assessments button 620 is activated. FIG. 6c shows a summary of the assessment status for the selected patient. The selected patient's name and room number are shown at block 632. A list of all available assessments is provided below block 632. Assessments that are activated for the current survey are shown as buttons. In the illustrated example, the wound, demographics, surface and Braden risk assessments are activated by selecting buttons 634, 636, 638, and 640, respectively. Assessments that are not activated for the selected survey are shown without buttons and with a status 648 of “N/A”, at block 642. The activated assessments are selected during the process of creating a survey, step 200 of FIG. 2, discussed above.

Available assessments have associated with them a status 648 of either “?” or “Ok” in the illustrated embodiment. If the status 648 is shown as “?”, the assessment has not yet been completed for the selected patient. Likewise, if the status 648 is shown as “ok”, the assessment has been completed. As shown in FIG. 6c, the wounds, demographics, surface, and Braden risk assessments are activated in the selected survey but have not yet been conducted on the selected patient.

FIGS. 6d, 6e, and 6f show screens that are displayed if the wounds button 634 of FIG. 6c is activated to conduct a wound assessment on the selected patient. In FIG. 6d, a table 650 will list all of the wounds reported by the patient, and the location of the wound on the body (“sub-site”), the stage of the wound, and a status indicator (column “S”) indicating whether the assessment has been completed for the reported wound. At block 652, the total number of wounds reported for the patient is displayed.

In FIG. 6e, a three-dimensional graphic image of the human body 656 is provided to assist the survey taker in defining the area of the body where a wound exists. The survey taker can rotate the image, for example by selecting right or left arrows 658 with a stylus, mouse, keyboard, or other suitable input device, to see different views of the body. The user selects an area of the body, e.g., by pointing with a stylus or mouse, to the location on the image 656 where a wound is located. In response, one or more lists of descriptions of specific body parts in the selected area of image 656 are provided, and the survey user selects the appropriate identifiers for the selected body part from these lists. For example, if the survey taker selects the right thigh of image 656 in FIG. 6e, the appropriate identifiers are “anterior right leg” and “thigh” as shown in FIG. 6f, blocks 660 and 662 (as illustrated, “site” and “sub-site”).

FIG. 6f shows a screen for entering additional information to complete the wound assessment for the selected patient, including wound site 660 and sub-site 662, discussed above, wound type 664, date wound acquired 666, stage of the wound 668, source of the wound 670 (e.g., “present when admitted”), information about documentation of the wound 672, topical information 674, objective information 676 and outcome information 678. When all of the requested information has been input for the patient's wound, the wound assessment is complete and the status 648 will be changed to “ok” on FIG. 6c.

When the demographics button 636 is selected (FIG. 6c), patient demographic information is recorded as shown in FIG. 6g. The demographic information collected is the same as is collected during the add patients step 210 of FIG. 2. In this case, the patient demographic information is input during the survey, instead of being pre-entered prior to conducting the survey as discussed above. FIG. 6c shows a display suitable for a handheld device; in order to input all of the patient demographic information, it is understood that multiple demographics screens may be presented sequentially and accessed by selecting the next button 680.

If a surface assessment is to be conducted, the surface button 638 of FIG. 6c is selected. A screen such as shown in FIG. 6h is displayed to conduct a surface assessment for the patient. A surface assessment is used to keep track of the particular support surface being used by the patient (e.g., make/model of support surface). This information is used to analyze whether a correlation exists between support surface and wound development. In FIG. 6h message line 682 indicates whether a surface has already been recorded for the patient. As illustrated, message line 682 indicates that the patient's support surface has not yet been recorded. The survey taker selects the support surface in use from a list of possible choices at block 684. If the patient's support surface is not available in the list, the add surface button 688 is selected to input the patient's support surface information. At block 686, the survey taker inputs the date on which the patient was put on, or began using, the indicated support surface.

FIGS. 6i, 6j, and 6k show example screens for completing the various types of wound risk assessments discussed above. FIG. 6i shows an input screen for conducting a Braden risk assessment. As illustrated, at block 690, a survey taker selects the appropriate responses to the survey criteria from lists of available choices defined for each survey criterion. When all of the survey criteria have been entered, computer program logic calculates the patient's risk of developing wounds according to Braden method, and displays the patient's risk score and assessment at block 692. As illustrated, the patient's risk score is 17 and risk assessment is low, based on the responses entered in block 690. FIGS. 6j and 6k show examples of similar screens for conducting Norton and Waterlow assessments, respectively. As in FIG. 6i, responses to the survey criteria for the selected assessment type are input at block 694 of FIG. 6j for the Norton assessment and block 698 of FIG. 6k for the Waterlow assessment. The patient's risk score and assessment using the Norton method is calculated and shown at block 696 of FIG. 6j and block 699 of FIG. 6k for the Waterlow assessment. Similar screens (not shown) are provided for conducting the Hill-Rom assessment and other “custom” assessments that may be added or defined by the facility. The above-described methods of wound risk assessment are well known by those of ordinary skill in the health care industry. See, for example, “Pressure Ulcer Risk Assessment Scales—the Missing Link,” by Kenneth Olshansky, M.D., published in Vol. 20, Issue 2 of Advances in Wound Care; “Risk Assessment Tools and Risk Factors,” AHCPR Supported Guidelines, National Library of Medicine Clinical Practice Guidelines; “Waterlow Pressure Sore Scores,” available at www.judywaterlow.fsnet.co.uk/wlow.htm; and “Prospective Cohort Study of Routine Use of Risk Assessment Scales for Prediction of Pressure Ulcers,” by Lisette Schoonhoven, et al., Vol. 325 of BMT (Oct. 12, 2002) (available at www.pubmedcentral.nih.gov).

FIGS. 6a-6k show screens suitable for implementing the process of conducting a wound survey of step 220 of FIG. 2. Some of the screens are shown in a form suitable for implementation on a handheld device, while others are shown in a form more suitable for implementation on a larger display screen, such as a microcomputer monitor. Of course, it is preferable to conduct the entire survey on the same computing device, whether handheld or otherwise. However, as discussed above, it is understood that the process of conducting a survey and the various assessments therein may be performed using any suitable input and output devices.

In the illustrated embodiment, the software application tracks how long each survey has been open. For prevalence surveys, which constitute a “snapshot” in time, the survey times out 24 hours after it was started and is marked either “complete” or “incomplete,” as appropriate. For incidence surveys, the survey state is reset each day and the previous day's survey is marked “complete” or “incomplete” as appropriate.

Referring now to step 230, once a user has completed a survey, the information is stored temporarily on second computing device 120 until it is transferred to a database of first computing device 100. As mentioned above, if second computing device 120 is a handheld device, a conduit is used to properly transfer the collected data from second computing device 120 to first computing device 100 as described above. If data is to be transferred to an external data storage 130 at a location 50 as described above, patient-identifying information is removed from the collected survey data prior to transfer. Prior to the transfer of completed survey data, the completed survey may be “closed” by programming logic so that no further data can be entered for the survey.

At step 240 of FIG. 2, an administrator or survey taker generates one or more reports based on the collected survey data in order to analyze the survey results. In the illustrated embodiment, a user may choose to analyze survey results, for example, to trace the history of a specific patient over time (e.g., from admission to discharge), break down data by department to review performance, analyze wound prevalence, incidence, or occurrence by demographic, evaluate operational efficiency, measure support surface purchase return on investment, and/or better manage patient outcomes.

An example of a currently commercially available reporting software is Crystal Reports, however, any suitable reporting tool may be used to generate the reports. In the illustrated embodiment, a user may select from a plurality of standardized reports (such as reports based on prevalence, incidence, and occurrence percentages, demographics such as age and admitting diagnosis, and surface assessments) or create one or more custom reports that show information of particular interest to the facility.

The available reports provide information on wound prevalence, incidence, and occurrence by patient or patient grouping (e.g., facility or department within a facility), enable tracking of wound progression by patient or patient grouping, correlate wound development with other possibly relevant factors such as the patient's age, previous location (where admitted from), support surface, wound location, treatment or therapy used on the patient, or any of the other patient demographics or wound criteria being tracked, and summarize risk assessment information for specified groupings of patients.

For instance, wound incidence tracking reports include, for a patient, the number and type of wounds acquired during the patient's stay at the facility, the number of such wounds healed, the support surface in use when a wound is acquired or healed, the length of time to acquire a wound, and the length of time required to heal a wound. Additionally, the support surface in use can be monitored by department (or other patient grouping) or by wound stage. Also, patients can be “ranked” by whether they have a very high, high, moderate, or low risk of developing wounds. Further, the risk assessment score can be broken down by patient grouping, for example, the number of patients at each risk level can be determined for each department.

Still further, the location of the wound on the body can be tracked. In the illustrated embodiment, the wound location, wound stage, patient grouping (e.g., hospital department) and support surface are tracked to enable a caregiver or health care provider to determine the number of wounds reported by patients using different support surfaces, the number of wounds in each stage that have been observed on patients by support surface, and wounds by stage for patients by department. Illustratively, wound stage is indicated as stage I, II, III, IV and or “necrotic,” as is well known in the art. The illustrated embodiment provides wound staging capabilities as defined by WOCN, NPUAP, and AHRQ. It is understood, however, that other suitable wound classification systems may also be used.

In the illustrated embodiment, such reports are generated in table form, as shown by the example Table 1 below, however, it is understood that any other suitable report form may be used.

TABLE 1
FACILITY A - DEPT. 101 - SUMMARY

| | |
|---|---|
| WOUND | SUPPORT SURFACE |

| STAGE | SURFACE A | SURFACE B | SURFACE C |
|---|---|---|---|
| I | 3 patients | 5 patients | 2 patients |
| II | 2 patients | 0 patients | 1 patient |
| II | 5 patients | 1 patient | 1 patient |
| IV | 1 patient | 0 patients | 1 patient |
| Necrotic | 0 patients | 0 patients | 0 patients |

Generally, in order to obtain a report, a user will input the report parameters by selecting a survey and patient grouping (e.g., facility or department) to be the subject of the report. A date range is also specified, for example, if the report relates to an incidence or occurrence study.

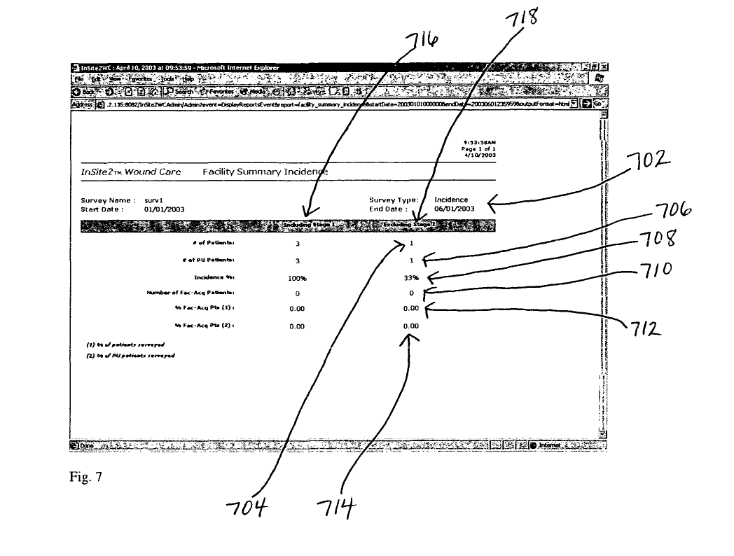

FIG. 7 shows an example of an incidence study summary for a facility. The report parameters, e.g., survey name, start and end dates (if applicable), and survey type, are displayed at block 702. As illustrated, the selected survey is of the incidence type, therefore, a date range is specified. The report detail is displayed below block 702. The information listed in the illustrated report includes the number of patients surveyed using the selected survey during the date range 704, the number of patients 704 that have wounds 706, the calculated incidence percentage 708, and, from the total number of patients in the study 704, the number of patients with wounds acquired while at the facility 710, the percentage 712 of patients 710 of the total number of patients surveyed 704, and the percentage 714 of patients 710 that have wounds as a percentage of the total number of patients surveyed having wounds 706. As shown in FIG. 7, this information is presented by wound stage, e.g., the first column 716 shows the results for all patients, including patients with wounds in stage I, and the second column 718 shows the results for all patients except for patients having wounds in stage I. In the illustrated embodiment, the incidence percentage is calculated by dividing the total number of patients with wounds by the total number of patients surveyed at the beginning of the study.

At step 250 of FIG. 2, survey results and/or the analysis thereof are reported to caregivers, health care providers, or others desiring such information. Such reporting is generally accomplished by transmitting reports generated at step 240 to parties who have requested such reports, by electronic mail, file transfer protocol, fax, phone or other suitable data communication method known in the art.

In certain of the illustrated embodiments, collected survey data from multiple facilities is stored in a centralized data repository (such as is shown in FIGS. 1e and 1f). In these embodiments, authorized users may view portions of the data from other facilities and compare their facility's results to others across the state, region, country, group of countries, or world. These comparisons may be used by the facility, for example, to identify trends and/or define performance benchmarks.

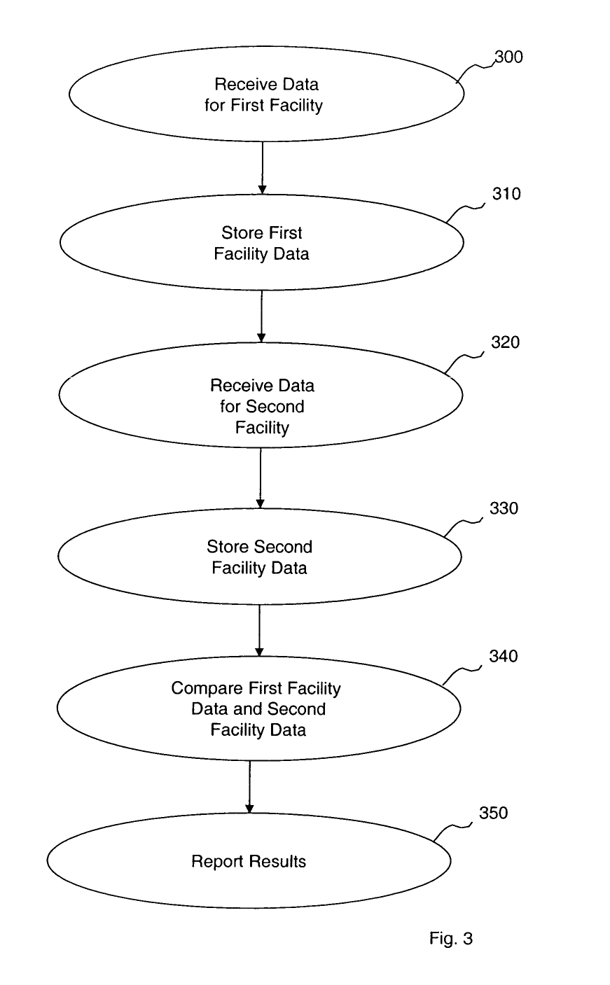

FIG. 3 shows a flow diagram of a method of the present invention, implemented in a computer system, for managing survey data collected at multiple facilities. The illustrated method is implemented using internet, web server, XML, SQL, and Informatical technology, however, it is understood that any other suitable computer, network, and database technology known in the art may be used. At step 300, data for completed surveys of a first healthcare facility 10, 12, or 14 is extracted from the local database of first computing device 100 and transmitted to a computer system at external location 50 over an external communications network, such as the Internet, using a secure communications protocol like HTTPS. A server computer programmed to check for newly received communications at location 50 periodically checks for new files. If the presence of a new file is detected, the new file is received at location 50 through an appropriate security firewall known in the art.

At step 310, survey data for first facility 10, 12, or 14 is stored in data storage 130 using data import techniques known in the art. As described in the attached exhibits, data storage 130 is preferably a database or data warehouse as known in the art, having a table structure that is substantially similar to the structure of the corresponding database at first computing device 100. Data storage 130 is capable of storing survey data (including facility data, survey name, survey time period, patient information, wound information, Braden/Norton/Waterlow/Hill-Rom assessment data, and bed/surface data) for one or multiple facilities. In the illustrated embodiment, an electronic notification, such as an email message, is generated and sent to a designated human or software agent, when the data transfer to data storage 130 is complete.

At steps 320 and 330, an essentially identical process as described above for steps 300, 310 is repeated for each additional facility 10, 12 or 14 from which survey data is received.

At step 340, a software process is triggered to create a cross-facility (e.g., regional or national) survey result by combining the data of multiple facilities, or to generate a cross-facility report. An example of such a report includes a comparison of the support surfaces used at different facilities and the corresponding prevalence or incidence of wounds. In another example, wound assessment data may be aggregated and averaged for all participating facilities (e.g., on a national basis). Then, each particular facility can compare its own assessment data to the cross-facility or national average values. For example, facility “A” may be one of 50 healthcare facilities that subscribe to the wound assessment system. At step 340, facility “A” can compare its wound ratio to the average wound ratio for all 50 facilities. However, due to the need to protect the confidential information of individual facilities, facility “A” is not able to view the specific data for any other facility. Such reports are generated using data reporting techniques known in the art. Users can view or print any of the various reports, examples of which are shown in Exhibit C. “Users” as used in this context may include persons or entities who do not complete surveys but are given access to the aggregate database. Such users' access is appropriately restricted to protect facilities' confidential information.

In another embodiment, a recommendation can be generated based on the survey results, for example, a recommendation for a change in treatment of a patient, or a change in the support surface used by patient(s). The recommendation may be communicated to a user, such as healthcare facility personnel or a support surface manufacturer, by email, fax, or other suitable communication means known in the art. An authorized user may review an automatically generated recommendation on-line and accept, edit, or reject the recommendation.

The Hill-Rom® Insite2™ Wound Care User's Guide, published April 2003 and incorporated herein by this reference, contains additional details relating to certain of the disclosed embodiments. Although the invention has been described in detail with reference to certain exemplary embodiments, it is understood that variations and modifications exist with the scope and spirit of the present invention as defined and described in the appended claims.

1. A computer program product embodied in an computer readable medium, the computer program product comprising instructions that when executed cause a computing device to be operable as a device for monitoring the development of wounds by patients of a health care facility, the device operating to:
display a survey to be conducted on patients of the health care facility, the survey including at least one wound risk assessment selected from a plurality of possible wound risk assessments,
display information about a patient for whom the survey is to be conducted,
receive survey data for the patient periodically during the patient's stay at the facility, and
display a risk score for each wound risk assessment in the survey based on the survey data for the patient.

2. The computer program product of claim 1, wherein the plurality of possible wound risk assessments includes Braden, Norton, Waterlow, and Hill-Rom risk assessments.

3. The computer program product of claim 2, wherein the plurality of possible wound risk assessments further includes a custom risk assessment for the facility.

4. The computer program product of claim 1, wherein the computing device is a handheld device.

5. The computer program product of claim 1, wherein the computing device further operates to display a plurality of available surveys from which a desired survey is may be selected.

6. The computer program product of claim 1, wherein the computing device further operates to store the survey data in a memory.

7. The computer program product of claim 1, wherein the survey is user-definable.

8. The computer program product of claim 1, wherein the survey is pre-defined by the facility.

9. The computer program product of claim 1, wherein the survey is configured to be conducted on patients that have a wound and on patients that do not have a wound.

10. The computer program product of claim 1, wherein the survey is configured to track the development of wounds by patients who do not have wounds at the time they are admitted to the facility.

11. A method implemented on a computing device, for studying the development of wounds by patients of a facility, the method comprising the steps of:
receiving a plurality of first survey data for a first facility over a network, the first survey data including wound information and support surface information for patients of the first facility,
storing the first survey data in a memory, and
analyzing the first survey data to determine whether there is a relationship between the wound information and the support surface information.

12. The method of claim 11, wherein the wound information includes an indication of whether patients have wounds.

13. The method of claim 11, wherein the support surface information includes the name of a support surface.

14. The method of claim 11, further comprising the step of communicating the results of the analyzing step to a person having a relationship with the facility.

15. The method of claim 14, wherein the person is affiliated with a support surface manufacturer.

16. The method of claim 11, further comprising the steps of:
receiving a plurality of second survey data for a second facility over a network, the second survey data including wound information and support surface information for patients of the second facility, storing the second survey data in a memory, and comparing the second survey data to the first survey data.

17. The method of claim 16, wherein the first and second survey data include assessment values, further comprising the step of averaging the assessment values of the first and second survey data.

18. The method of claim 11, wherein the first survey data includes support surface information for patients that do not have a wound.

19. The method of claim 11, wherein the analyzing step includes calculating a wound incidence percentage.

20. The method of claim 19, wherein the support surface information includes an indicator of a support surface used by each patient, and the analyzing step includes determining a wound incidence percentage for each support surface in use.

21. The method of claim 11, wherein the wound information includes an indicator of wound stage, the support surface information includes an identifier for each support surface in use, and the analyzing step includes determining the number of wounds in each stage by support surface.

22. A computer program product embodied in a computer-readable medium, the computer program product comprising instructions that when executed cause a computing device to be operable as a device for monitoring the incidence of wounds in patients of a health care facility, the device operating to:
receive a plurality of wound information for a grouping of patients of the facility, the wound information including an indication of whether a patient has a wound,
compute a wound incidence percentage for the grouping of patients, and
communicate the wound incidence percentage to a person associated with the facility.

23. The computer program product of claim 22, wherein the wound incidence percentage is computed over a time period equal to the average patient stay.

24. The computer program product of claim 22, wherein the wound incidence percentage is computed for each patient for a time period beginning on an admission date and ending on a discharge date.

25. The computer program product of claim 22, wherein the computing device further operates to receive information about an activity level of the facility.

26. The computer program product of claim 25, wherein the computing device further operates to analyze the activity level information and the wound incidence percentage.

27. The computer program product of claim 25, wherein the computing device further operates to conduct an occurrence study.
